(19) United States
(12) Patent Application Publication
(10) Pub. No.: US 2012/0071867 A1
(43) Pub. Date: Mar. 22, 2012

(54) DIODE LASER SYSTEMS AND METHODS FOR ENDOSCOPIC TREATMENT OF TISSUE

(75) Inventors: Andrew Ryan, Atherton, CA (US); Ramsey Shanbaky, San Francisco, CA (US); Pankaj Jay Pasricha, Cupertino, CA (US)

(73) Assignee: Metalase, Inc., San Jose, CA (US)

(21) Appl. No.: 13/051,891

(22) Filed: Mar. 18, 2011

Related U.S. Application Data
(60) Provisional application No. 61/315,338, filed on Mar. 18, 2010.

(51) Int. Cl. A61B 18/22

(52) U.S. Cl. 606/15; 606/16

(57) ABSTRACT

Embodiments of the present invention provide improved laser systems and methods for endoscopic laser treatment of abnormal tissue such as abnormal mucosal tissue, for example esophageal dysplasia that is also referred to as Barrett's esophagus (BE). The systems and methods described here can also be used in many applications where treatment of shallow surface layers with minimal damage to the tissue beneath is desirable, for example BE tissue comprising hemoglobin. In many embodiments, the system is configured to emit light energy having an optical wavelength to treat tissue having oxygenated hemoglobin.

This application claims the benefit of Provisional Application No. 61/315,338 (Attorney Docket No. 028388-000100US), filed on Mar. 18, 2010, the full disclosure of which is incorporated herein by reference.

### BACKGROUND OF THE INVENTION

The present invention is directed to the treatment of tissue with diode lasers, in particular endoscopic treatment of tissue comprising hemoglobin. Although specific reference is made to the treatment of Barrett's esophagus, embodiments can be used to treat many tissues, for example tissue comprising hemoglobin.

Prior systems and methods to treat tissue pathology with laser systems are less than ideal. In at least some instances, the laser systems can be complex and less effective in treating tissue having pathological lesions than is desirable. Also, at least some laser systems provide light energy that is less than ideal for the treatment of some tissues, for example esophageal tissue having hemoglobin. In at least some instances, the absorption of the laser energy by the target tissue can be insufficient to prevent the penetration of laser energy to relatively greater depths, such that energy may be inappropriately delivered to healthy tissue at depths below the target tissue in some instances. In at least some systems and methods, laser energy can be scattered away from the beam near the surface of the target tissue such that insufficient treatment energy may be delivered to the targeted tissue near the surface.

With the treatment of Barrett's esophagus, for example, the lesion may correspond to the upper mucosal layer located above a lamina propria layer, and in at least some instances treatment may result in tissue damage below the lamina propria such as damage to one or more of the muscularis mucosae or the muscularis externa/propria layers located 1 mm or more below the surface of the target tissue.

Work in relation to embodiments of the present invention also suggests that tissue absorption that is somewhat greater than would be desired may result in a less than ideal treatment in at least some instances. As the targeted tissue having the lesion may have a substantial thickness light absorption that is too strong can result in decreased light penetration and less effective treatment to a lower portion of the lesion than would be ideal.

Although electrodes can be used to treat pathological tissue such as dysplasia, the heat energy generated with electrodes may not be tissue specific, such that in at least some instances the treatment can damage healthy tissue. The damage to healthy tissue may result in recovery times that are somewhat longer than would be ideal in at least some instances. Also, treatment that is not tissue specific may result in an incomplete treatment such that the treatment outcome can be less than ideal in at least some instances.

In light of the above, it would be desirable to provide improved systems and methods for the treatment of tissue, and in particular for the improved treatment of tissue having hemoglobin such as Barrett's esophagus.

Embodiments of the present invention may be used for the treatment of other conditions where the removal of tissue is associated with a desirable clinical outcome.

### BRIEF SUMMARY OF THE INVENTION

Embodiments of the present invention provide improved laser systems and methods for endoscopic laser treatment of abnormal tissue such as abnormal mucosal tissue, for example Barrett's esophagus (BE) which may include esophageal dysplasia and metaplasia. The systems and methods described here can also be used in many applications where treatment of shallow surface layers with minimal damage to the tissue beneath is desirable, for example BE tissue comprising hemoglobin.

In many embodiments, a medical laser system is configured with an optical wavelength to treat tissue having oxygenated hemoglobin. In many embodiments, the laser system can treat tissue having Barrett's esophagus such that the layer having Barrett's esophagus is removed with ablation and the underlying lamina propria can be coagulated to inhibit bleeding. The laser beam energy and emitted wavelength can be particularly well suited for treatment that includes a boundary of the Barrett's esophageal tissue. In many embodiments, the wavelength of light is within a range from about 400 nm to about 430 nm, near the peak absorption of oxygenated hemoglobin. The Barrett's tissue including the tissue boundary can be ablated substantially, and the healthy squamous layer along the tissue boundary and the lamina propria under the healthy squamous layer have substantially less absorption of light energy and corresponding damage, such that the patient can recover faster and allow for complete treatment of the Barrett's tissue layer. The medical laser system may comprise a laser diode module having one or more laser diodes configured to emit light energy near an absorption peak of oxygenated hemoglobin, for example near the absorption peak of 416 nm. The one or more laser diodes can be coupled to at least one optical fiber, such that a light flux energy within a range from about 200 W/cm² to about 400 W/cm² can be delivered to the tissue with a cumulative amount of energy delivered within a range from about 100 J/cm2 to about 300 J/cm2, such that the pathological tissue having Barrett's esophagus can be ablated and underlying tissue, particularly the lamina propria, can be coagulated. The treated tissue may comprise a first portion removed with ablation and a second coagulated portion. The one or more laser diodes may comprise a plurality of laser diodes coupled to a plurality of optical fibers, and the light energy from the plurality of optical fibers can be combined so as to provide a smoothed laser beam energy profile distribution. The plurality of optical fibers can be coupled to a mono-mode or multi-mode optical delivery fiber, and the delivery fiber can be inserted into the patient. The distal end of the optical fiber inserted into the patient can be scanned with movement of the distal end so as to treat an area of the patient substantially larger than the distal end of the optical fiber.

### BRIEF DESCRIPTION OF THE DRAWINGS

FIGS. 1A-1C show an overview of the system and its use in an endoscopic procedure, in accordance with embodiments of the present invention;

FIG. 2A shows a diagram of a cross-section of a normal esophageal wall;

FIGS. 2B-2C show microscope views of a cross-section of a normal esophageal wall;

FIG. 2D shows an image of Barrett's esophagus viewed through an endoscope;

FIG. 3 shows a hemoglobin absorption spectra for oxygenated hemoglobin (HbO₂) and non-oxygenated hemoglobin (Hb) in accordance with embodiments of the present invention;

FIG. 4 heat flux versus depth and wavelength, in accordance with embodiments of the present invention;

FIG. 5 shows a laser system schematic, in accordance with embodiments of the present invention;

FIG. 6 shows a cross section of a delivery fiber, in accordance with embodiments of the present invention;

FIGS. 7A-7B show the delivery fiber placed in the esophagus during treatment, in accordance with embodiments of the present invention;

FIG. 8A shows a coupler in an off-position and an on-position, in accordance with embodiments of the present invention;

FIG. 8B shows an aim diode for use in a coupler, in accordance with embodiments of the present invention;

FIGS. 9A-9B show selective ablation of a Barrett's esophagus lesion based on selective absorption of oxygenated hemoglobin, in accordance with embodiments of the present invention;

FIG. 10 shows a microscope view of treated portions of small porcine intestine treated at two different flux levels, in accordance with embodiments of the present invention;

FIG. 11 shows a microscope view of a treated porcine esophagus, in accordance with embodiments of the present invention;

FIG. 12 shows a section of the porcine esophagus treated with the laser system as described herein and such that there is an absence of mucosa as a result of laser thermal ablation, in accordance with embodiments of the present invention;

### DETAILED DESCRIPTION OF THE SPECIFIC EMBODIMENTS

Embodiments as described herein can exploit the absorption near absorption peaks of at least one type of molecule found within the human body, for example hemoglobin. As used herein, a tissue treatment region encompasses an ablation layer and a coagulation layer.

Embodiments of the present invention provide improved laser systems and methods for endoscopic laser treatment of abnormal tissue such as abnormal mucosal tissue, for example esophageal dysplasia that is also referred to as Barrett's Esophagus. The system and method described here can also be used in many applications where treatment of shallow surface layers with minimal damage to the tissue beneath is desirable, for example tissue comprising hemoglobin.

Barrett's esophagus (BE) is a disorder in which the lining of the esophagus, the tube which carries food from the throat to the stomach, is damaged. The normal squamous epithelium lining of the esophagus is replaced by metaplastic columnar epithelium, which is a cell type more typically found in more distal parts of the gastrointestinal system. It is generally thought that the damage to the esophageal lining is caused by chronic exposure to stomach acids, such as may occur from acid reflux. Patients with BE may develop dysplasia, which increases the risk of developing esophageal cancer, such as esophageal adenocarcinoma, a particularly lethal cancer. As cases of Barrett's esophagus have increased in recent years, improved methods and systems for treatment of such conditions is desirable.

Embodiments of the present invention can be used for the treatment of BE. Barrett's esophagus is typically associated with gastroesophageal reflux disease (GERD) whereby the reflux of gastric fluids into the esophagus from the stomach transforms the lining of the esophagus. An abnormal Barrett's tissue is typically pinkish or reddish in color compared to the normal whitish color of healthy tissue lining an esophagus. The reddish or pinkish color of the abnormal lining contains a higher density of hemoglobin and will therefore have a higher absorbance at the diode laser wavelength leading to more effective ablation of the abnormal tissue. Further, the difference in color is readily observable through endoscopic observation thus the treatment area can be limited to only those regions where the abnormal tissue actually occurs with a spatial precision equal to the beam diameter emerging from the fiber.

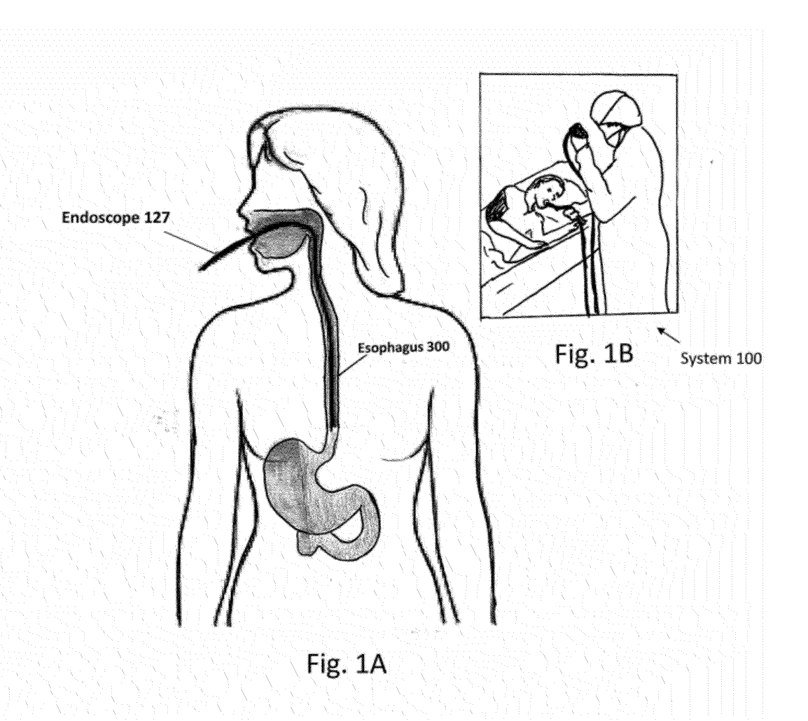

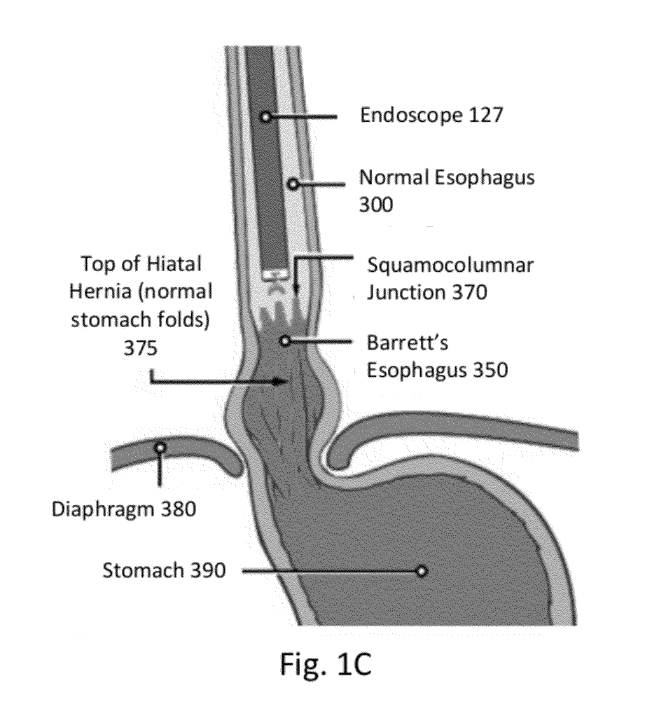

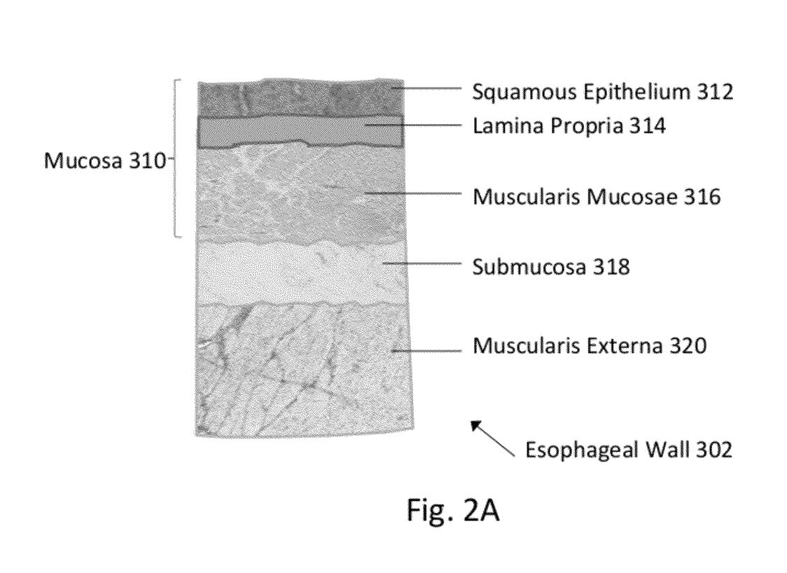

FIG. 1A shows an endoscope 127 inserted through the esophagus 300 of a patient through which system 100 may be used to treat abnormal mucosal tissue on the lining of the esophagus, and in particular Barrett's esophagus. FIG. 1B shows a patient reclined in a bed while a physician utilizes system 100 to treat the patient in an endoscopic procedure, the endoscope having been inserted down the throat of the patient into the esophagus. The endoscope allows the physician to view the esophageal lining during treatment with system 100. FIG. 1C shows an endoscope 127 of system 100 extending into the esophagus 300 just above of the squamocolumnar junction 370 at which the BE lesions 350 begin. In this example, the BE lesions 350 are located near a hiatal hernia 375 above the diaphragm 380 and stomach 390.

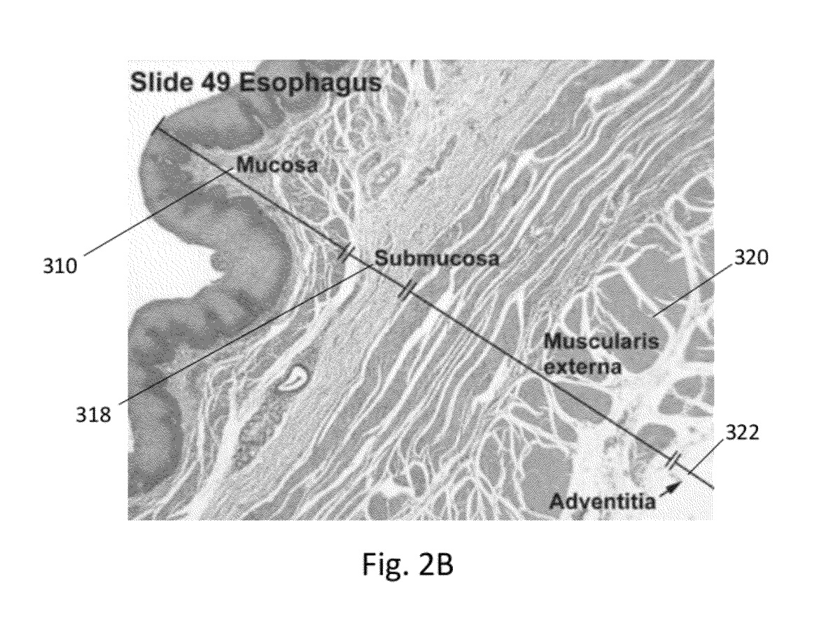

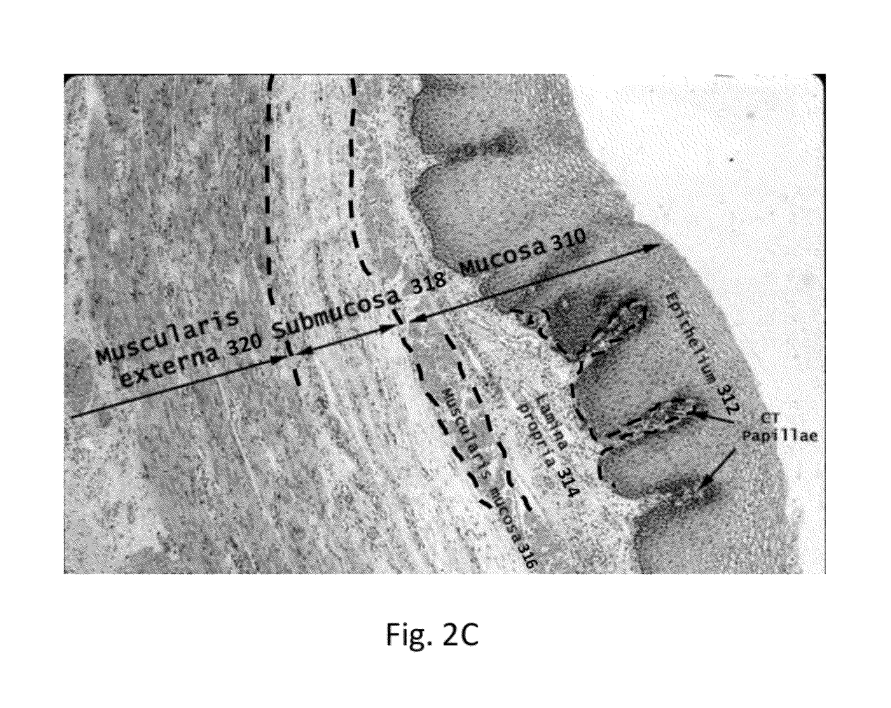

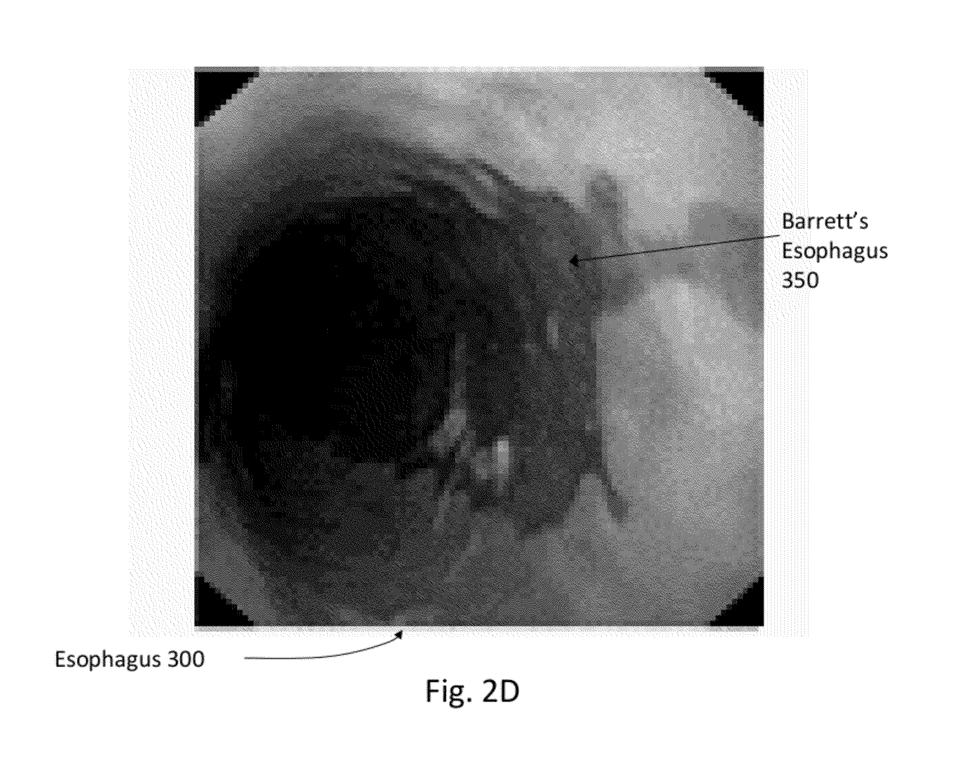

FIG. 2A shows a cross-sectional diagram of a normal esophageal wall 302. The normal esophageal wall 302 comprises three main layers, the mucosa 310, the submucosa 318 and the muscularis externa 320. The mucosa 310 varies in thickness from about 1500 μm to 2000 μm and comprises three primary layers: the squamous epithelium 312, the lamina propria 314 and the muscularis mucosae 316. The muscularis mucosae is typically about 1000 μm thick, while the combined thickness of the squamous epithelium 312 and the lamina propria 314 varies between 500 μm and 1000 μm. The lamina propria 314 acts as a barrier between the muscularis mucosae 316 layer and the squamous epithelium 312 layer. The squamous epithelium 312 layer, which is generally one-cell thick, lines the inside wall of a normal healthy esophagus. In a BE lesion, metaplastic columnar epithelium cells from the gastrointestinal system replace the squamous epithelium 312 cells. Generally, the BE lesions are isolated to the squamous epithelium 312 layer, although in some cases BE lesions have extended into deeper regions. The above layers can also be seen in FIGS. 2B and 2C, which show microscope slide images a normal esophageal wall 302 cross-section. As shown in the endoscopic image in FIG. 2D, BE lesions are typically located with an endoscope. Since BE lesions comprise substantially more hemoglobin than healthy squamous epithelium, and hemoglobin is the iron-containing oxygen-transport metalloprotein in red blood cells, the BE lesions typically appear pinkish or reddish in comparison to healthy lining in an esophagus 300. As is evident from FIG. 2D, the pinkish or reddish BE lesions 350 are clearly evident contrasted against the relatively pale, whitish lining of the esophagus 300. The presence of BE lesions is typically confirmed via biopsy.

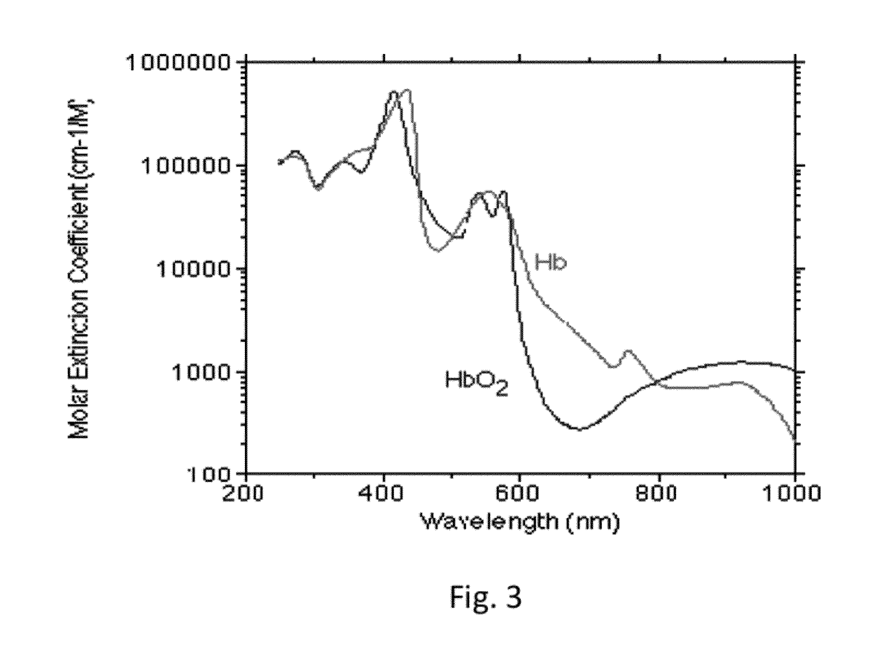

The presence of the hemoglobin in the BE lesions 350 allows the system 100 to use light energy to treat the abnormal tissues by exploiting the absorption spectra for hemoglobin. FIG. 3 shows the hemoglobin absorption spectra for oxygenated hemoglobin (HbO₂) and non-oxygenated hemoglobin (Hb). The average peak absorption wavelength for hemoglobin is about 420 nm, the peak absorption for non-oxygenated hemoglobin (Hb) being 433 nm while the peak absorption for HbO₂ is 416 nm.

In many embodiments, a medical laser system is configured to emit light energy from diode lasers of a diode laser module. The light energy emitted from the laser diodes of the laser diode module can be used to treat tissue near the 420 nm absorption peak of hemoglobin, for example 405 nm light energy within about 20 nm of the 420 nm peak, and nearer the 416 nm absorption peak of oxygenated hemoglobin, such that tissue comprising hemoglobin can be selectively treated with the light energy emitted from the laser diodes. The system can be configured to pulse the laser diodes to generate a pulsed laser beam to treat the tissue, for example with ablation of the tissue. The auto absorbance of the tissue comprising hemoglobin can allow a thin layer of a first pathological tissue comprising hemoglobin to be treated, and non-pathological tissue comprising less hemoglobin is less damaged with the beam exposure, such that the pathological tissue can be treated. The system may comprise an adjustable laser beam pulse intensity and duration to treat selectively the targeted tissue.

The treatment of tissue as described herein may comprise deposition of energy sufficient to substantially alter the tissue so as to achieve a desired therapeutic benefit. For example, the treatment may comprise heating the tissue so as to coagulate the tissue, and in many embodiments the tissue can be ablated. Ablation of tissue as described herein encompasses removal of the tissue, for example with heating of the tissue to 100° C. so that water comprising the tissue vaporizes.

In the visible light region, the absorption of the hemoglobin molecule can be exploited. Embodiments as described herein can exploit the largest hemoglobin absorption features near 420 nm, and the wavelengths used can be within about +/−50 nm of the peak, for example +/−20 nm of the peak and may comprise a wavelength of about 405 nm from a gallium-nitride (GaN) laser diode. Several of these GaN diode laser devices can be combined, for example ganged together, and used to produce higher power modules. The maximum power available from a commercially available single GaN laser diode operating near 405 nm is currently specified as 700 mW, although there have been demonstration devices of 3000 mW at 450 nm. The fiber-coupled power available around 405 nm from the combined fibers in a 20-emitter module can be 10 W. In this module, each emitter can be coupled into an independent 125 μm (numerical aperture 0.2) diameter fiber, which are then collected together to fill a 650 μm aperture.

The physics of the laser-tissue interaction can be combined in accordance with the embodiments as described herein. The laser beam intensity is affected by both absorption and scattering as it propagates through the tissue. In the first order, the intensity of the electromagnetic radiation as a function of depth in tissue is governed by Lambert's Law for turbid media, as follows:

I(z)=I₀exp[−(α+αₛ)z] (EQ. 1)

Where z is the depth in the tissue, I₀ is the optical intensity at z=0, α is the absorbance of the medium and αₛ is the scattering coefficient of the medium. The wavelength-dependence of the tissue response enters through the wavelength dependence of the absorbance and the scattering coefficient. The optical penetration depth, Lₒ is determined from the inverse of these two parameters, as in the following equation:

Lₒ=1/(α+αₛ) (EQ. 2)

Lₒ corresponds to the depth in the tissue at which the optical intensity is reduced to 1/e of its value at the surface.

A more sophisticated analysis incorporating the effect of the absorption of scattered light, called the Diffusion Approximation, separates the laser intensity into coherent, I_{c}, and diffuse, I_{d} components.

I=I_{c}÷I_{d}32 I_{d0}exp[−(α+αₛ)z]+I_{d0}exp(−α_{d}z) (EQ. 3)

This simplified expression suppresses the z-dependence of I_{d0} but captures the significance of the diffuse component. The effective attenuation of the diffuse component, α_{d}, is: [symbol]
The g parameter accounts for the directionality of the scattering where g=1 denotes purely forward scattering, g=0 denotes purely isotropic scattering and g=−1 denotes purely backward scattering. For tissue g≈0.90-0.95 for visible wavelengths.

Heat is generated inside the tissue during laser exposure by means of a two step process. First, photon energy is absorbed by the molecules that comprise the tissue. These molecules then decay from their excited state via inelastic collisions with particles in the surrounding medium. Therefore, the temperature rise originates microscopically from the transfer of photon energy to kinetic energy. In the first order, the heat flux in the medium is given by:

S(z,λ)=αI(z,λ)=I₀exp[−(α+αₛ)z] (EQ. 5)

The wavelength dependence of the tissue response enters through the wavelength dependence of the absorbance, α and scattering, αₛ. Generally, while both parameters may have roughly equal influence on the evolution of intensity, the absorption parameter dominates the generation of heat.

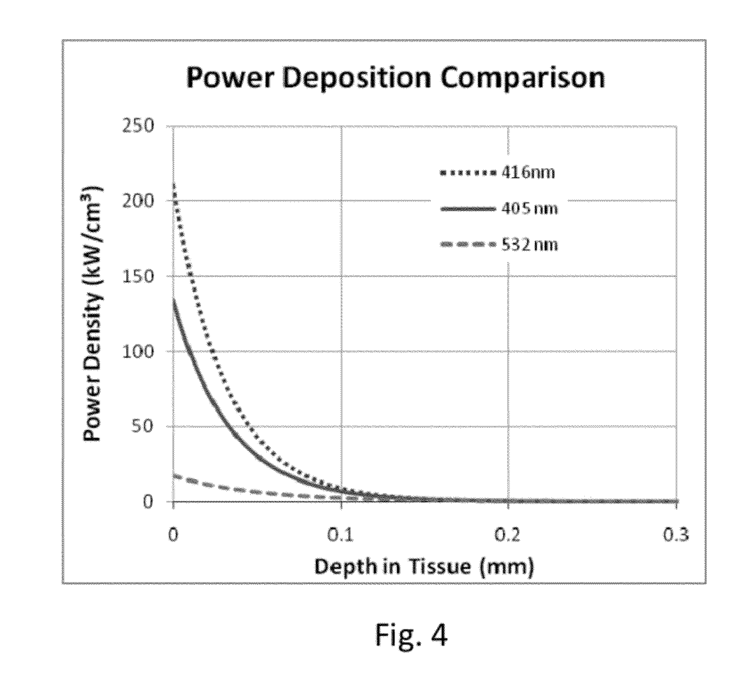

The relationship between these parameters is illustrated in FIG. 4 by comparing the heat flux provided by beams at different wavelengths, in particular 416 nm, 405 nm, and 532 nm for a 10 W, 650 gm diameter beam. For a 416 nm beam, the absorbance of oxygenated hemoglobin is approximately 2795 cm⁻¹ which is roughly 11.9 times larger than the 235 cm⁻¹ absorbance of oxygenated hemoglobin at 532 nm. For a 405 nm beam, the absorbance of oxygenated hemoglobin is approximately 1775 cm⁻¹ which is roughly 7.5 times larger than the 235 cm⁻¹ absorbance of oxygenated hemoglobin at 532 nm. For tissue comprised of 2.5% oxyhemoglobin these numbers are 69.9 cm⁻¹, 44.7 cm⁻¹, and 5.9 cm⁻¹ for the 416 nm absorption, the 405 nm absorption and the 532 nm absorption, respectively. The corresponding scattering coefficients measured in tissue are 245 cm⁻¹, 250 cm⁻¹, and 190 cm⁻¹ for the scattering at 416 nm, 405 nm, and 532 nm respectively. For a 10 W beam that uniformly fills a 650 μm aperture, I₀=3.01 kW/cm². Using these numbers, the heat flux can be graphed as a function of depth in tissue for the first order approximation (see FIG. 4). At each of the three wavelengths nearly all of the energy is deposited in the first 100 μm of tissue. The difference is that the much larger absorption at 405 nm and the 416 nm corresponds to significantly more energy being deposited by the 405 nm and the 416 nm beams. A Diffusion Approximation treatment would be qualitatively similar but show more energy deposition by the 532-nm beam at greater depths.

One method of using the first order approximation to calculate the conditions required for vaporization is to calculate the conditions required to vaporize the water in the tissue. The vaporization of water in tissue occurs in two steps. First, the temperature of the tissue being vaporized is heated to 100 C. Assuming that the specific heat of tissue is approximately equal to the specific heat of water, that the tissue begins at 37° C., and that the tissue is 80% water by weight, the heat required to raise a mass of tissue, m, to 100° C. can be calculated as Q₁ with the following equation:

Q₁=mcΔT=m(0.8)(4.3 J/g)(63 C)=m×217 J/g (EQ. 6)

Next, the heat required to vaporize that same mass of tissue can be calculated. The energy required to vaporize the tissue is the energy require to vaporize the water within the tissue, thus:

Q₂=mQᵥₐₚ=m(0.8)(2253 J/g)=m×1802 J/g (EQ. 7)

Given the assumption that the density of the tissue is approximately the same as that of water, 1 g of tissue would have a volume of approximately 1 cubic centimeter. The heat of vaporization per unit volume can then be calculated by the following equation:

Qₜₒₜₐₗ/V=(Q₁+Q₂)/V=2019 J/cm³ (EQ. 8)

Given the above definitions, the heat flux at a depth of one optical penetration depth, Lₒ, is considered using the following equation:

S(Lₒ,λ)=αI₀ exp[−(α+αₛ)Lₒ]=αI₀/e (EQ. 9)

Thus, for the 416 nm beam (10 W at 416 nm emitted from 650 μm core as described above), the volumetric flux is 72.4 kW/cm³ at the optical penetration depth of 33.9 μm. For the 405 nm beam (10 W at 405 nm emitted from 650 μm core as described above), the volumetric flux is 49.5 kW/cm³ at the optical penetration depth of 33.9 μm. At that same depth, the flux from a 532 nm beam of the same power and size would be only 9.1 kW/cm³ due largely to the difference in absorbances. At these respective fluxes, the 416 nm beam would take 27.9 milliseconds (27.9×10⁻³ s) to vaporize the tissue to its optical penetration depth and the 405 nm beam would take slightly more than 40 milliseconds (40×10⁻³ s) to vaporize the tissue to that depth, while the 532 nm beam would require more than 221 ms to vaporize to that same depth. More generally, the 416 nm beam with a 650 tm diameter can vaporize a 33.9 μm layer with just 279 mJ of energy and the 405 nm beam with a 650 μm diameter can vaporize the 33.9 μm layer with 408 mJ of energy, whereas a 532 nm beam with the same cross section requires 2210 mJ. In the latter case, the extra energy (1931 mJ of energy as compared to the 416 nm beam and 1802 mJ of energy as compared to the 405 nm beam) continues to penetrate and damage the tissue beneath the vaporization layer. Thus, the higher absorption provided by the 416 nm and the 405 nm beams allows for vaporization of tissue while reducing thermal damage to the tissue beneath. Moreover, vaporization of a given amount of tissue with the 405 nm beam requires less total energy than would the 532 nm beam. It should be noted that this calculation has been simplified by neglecting the effect of the absorption of scattered light. As before, the details will change but the basic conclusions will remain the same as these higher order effects are included.

The above example is meant to be illustrative. The same physics holds true for lower or higher power beams with larger or smaller diameters. Generally, a 405 nm light will vaporize a given tissue layer in less time than would a 532 nm light. Additionally, more excess energy will be delivered to deeper tissue by the more weakly absorbed wavelength. The above calculation has been simplified by neglecting the flow of heat away from the irradiated region. Heat flow affects the total amount of energy required to achieve vaporization, but the higher-absorbed wavelength, in this example the 405 nm beam, will typically require less energy to vaporize a layer of a given thickness than would a lower-absorbed wavelength. The same physics also determines the absorption and vaporization interaction at other wavelengths. In particular, the least amount of energy is required for a beam at approximately 420 nm for hemoglobin, approximately 433 nm for non-oxygenated hemoglobin and approximately 416 nm for oxygenated hemoglobin.

It should further be noted that in laser treatment of tissue, coagulation and necrosis typically occurs for tissue heated above 60 degrees Celsius. Using the same methodology as noted above, a 23 degree temperature rise would be required and the flux required to achieve coagulation can be calculated by the following equation:

Q₁=mcΔT=m(0.8)(4.3 J/g)(23 C)=m×79.1 J/g (EQ. 10)

In many applications, limiting the depth of tissue in which coagulation is produced is desirable; however, a more detailed thermal model may be required to make effective predictions about the conditions under which coagulation occurs at depths significantly greater than the optical penetration depth. Such a detailed thermal model may account for the lateral diffusion of heat as well as the diffusion of heat during the pulse.

It is desirable to minimize the coagulation layer in treating Barrett's patients as it may lead to negative side effects such as dysphagia and odynophagia. However, one benefit of the coagulation layer is that it provides hemostasis, which prevents bleeding following treatment. Lasers that penetrate even less than 405 nm, e.g. a diode laser operating at the 416 nm absorption peak, might not produce enough of a coagulation layer to provide hemostasis.

Another benefit of the lower power requirement of the 405 nm light is an increased allowable optical fiber bend radius in the optical fiber. Fiber bend radius is the radius at which an optical fiber can be safely bent without breaking. As the power in the fiber increases, the allowable fiber bend radius increases as well. Additionally, at shorter wavelengths, the spatial extent of the evanescent fields generated by the light inside the fiber is smaller resulting in less light energy being lost upon bending through the fiber. The combination of low power and short wavelength allows for fiber configurations having a bend radius less than the width of the esophagus, such that the light energy can be directed to a wall of the esophagus.

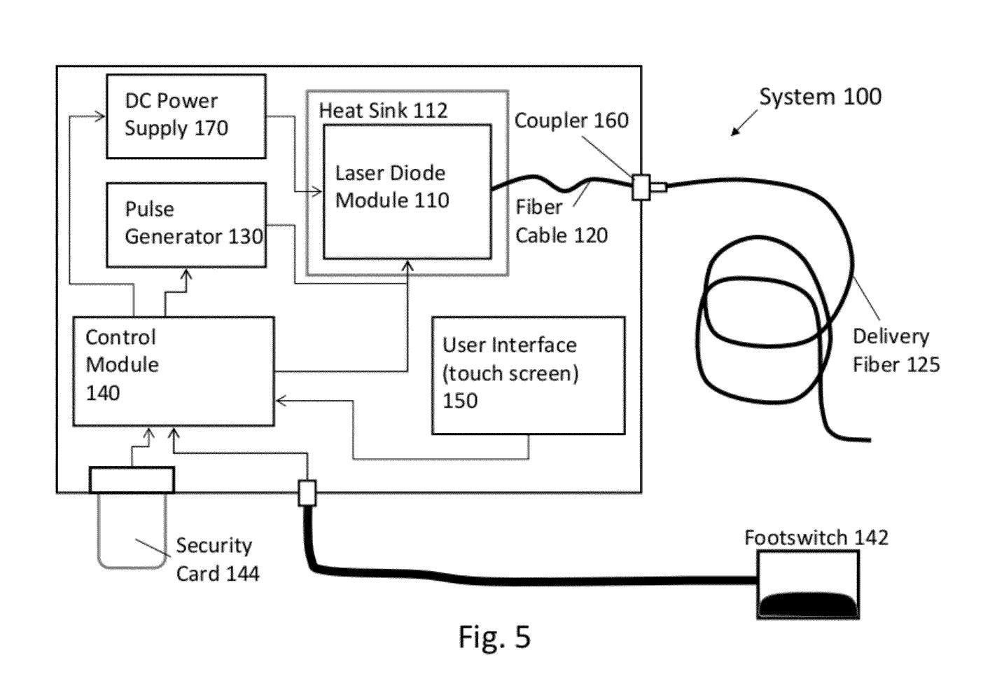

An exemplary embodiment of system 100 is shown in the schematic of FIG. 5. In this embodiment, system 100 comprises a laser diode module 110 disposed in a heat sink 112, the laser diode module 110 emitting light energy to fiber cable 120, which is optically coupled to a delivery fiber 125 for delivering the light energy to the tissue treatment region. Optionally, the delivery fiber 125 is optically coupled to fiber cable 120 by coupler 160. In this embodiment, the delivery fiber 125 is configured to be inserted down a throat of a patient to treat abnormal tissues in the esophagus, particularly BE lesions. The system 100 further comprises a DC power supply 170, a pulse generator 130, a control module 140, and a user interface 150, such as a touch screen. The control module 140 may be configured to receive user input from a control, such as a footswitch 142 for turning the laser treatment on and off and a user interface 150 for entering treatment parameters, and an authorization input, provided by a security card 144 input into a card reader.

In an exemplary embodiment, system 100 comprises a diode laser module 110. Diode laser module 110 includes at least one laser diode to emit light energy, but may include multiple diode lasers. Preferably, diode laser module comprises a plurality of diode lasers coupled to a plurality of optical fibers, and each diode laser can be coupled to a fiber such that the laser diodes are each individually coupled to at least one of the fibers. The one or more fibers can be collected together so as to comprise fiber cable 120. The system may comprise a control module 140 configured to send signals such as on or off signals and power level signals to the laser diode module 110. The signals can be received by the control module 140 from a footswitch 142 depressed by the user when the user wants to initiate treatment, for example from a temperature sensor which monitors the temperature of the laser module. The system may comprise a user interface 150 wherein the user enters pulse and power parameters that determine the treatment. The system may comprises a pulse generator 130 coupled to the control module 140, such that the pulse generator 130 receives signals from control module 140 so as to determine the duration, frequency and amplitude of the pulses sent to the laser diode module 110 to provide the pulse energy amount and duration of the laser beam. The system typically comprises a DC power supply 170 which provides energy to drive the diode laser module 110 and the other system modules. The system 100 may further comprise a user interface 150 wherein data from the user is entered and system data is displayed. The laser diode module 110, pulse generator 130, DC power supply 170, control module 140 and user interface 150 can be housed within a chassis.

In many embodiments, the fiber cable 120 can be connected to the chassis wall via a fiber connector or coupler 160, the fiber connector can provide efficient coupling of light energy from the fiber cable 120 to the delivery fiber 125. The chassis wall may include a connection port for the footswitch 142, and a connection where AC power may be delivered to the system. The chassis wall may include a USB port where a diagnostic tool may be connected to access system data. Optionally, the chassis wall may include a security-card reading port configured to receive security card 144 to ensure that only adequately designed delivery fibers 125 are used with the system.

Laser Diode Module

In an exemplary embodiment, system 100 comprises at least one laser diode module 110 to emit light energy, preferably at a wavelength suitable for absorption by an abnormal tissue to be used in treating the tissue. For example, to treat BE lesions, the at least one diode module 110 may emit light energy having a wavelength from about within a range from about 360 nm to about 450 nm, more preferably 400 nm to 430 nm, and even more preferably 400 nm to 410 nm. Typically, the output power of the system delivered to the tissue is at least about 10 W. The laser diode module 110 may comprise one laser diode optically coupled to an optical fiber or a bundle of fibers, or may comprise multiple diodes coupled to an optical fiber or a bundle of fibers for directing the light energy to the treatment site. In an exemplary embodiment, the optical light flux energy exiting the at least one optical fiber corresponds to an optical light flux energy on a surface of the tissue within a range from about 200 W/cm² to about 400 W/cm². In many embodiments, the optical light flux energy exiting the at least one fiber and optical light flux energy are each within the range from about 200 W/cm² to about 400 W/cm² when a distal end of the at least one fiber is placed within about 1 mm of the surface of the tissue.

In one embodiment, laser diode module 110 comprises the NUV101E slot module sold by Nichia corporation. The NUV101E includes 20 laser emitters coupled to individual fibers which are then combined at an optical connector into a 650 μm diameter fiber cable 120 with a numerical aperture of 0.2. The NUV101E emits light in the wavelength region between 400 and 410 nm, requires a 24 V and 4 A regulated DC power supply and voltage pulses up to 3.5 V. The typical maximum power output is at least about 10 W. The NUV101E returns a signal proportional to the submount temperature of the diode lasers. The NUV101E also has an LED that indicates the condition of the module: the LED shining green when the module has DC power, shining red when in a fault condition (either high temperature or a voltage signal that is too high) occurs, and off when no power is applied. The operating temperature of the laser diode module 110 is preferably between 20 and 30° C. In another embodiment, the laser diode module 110 may include two NUV101E slot modules and use polarization beam combining to produce a 20 W beam.

In some embodiments, the laser diode module 110 comprises the NUV102E slot module also sold by Nichia corporation. The NUV102E contains 4 laser emitters coupled to individual fibers which are then combined at the optical connector into a 250 μm diameter fiber cable with a numerical aperture of 0.2. The NUV102E emits light in the wavelength region between 400 and 410 nm, requires a 24 V and 1 A regulated DC power supply and voltage pulses up to 3.5 V. The typical maximum power output is 1.2 W. The NUV102E returns a signal proportional to the submount temperature of the diode lasers. The NUV102E has an LED that indicates the condition of the module: the LED shining green when the module has DC power, shining red when in a fault condition (either high temperature or a voltage signal that is too high) occurs, and off when no power is applied. The operating temperature of the laser diode module 110 is preferably between 20 and 30° C.

In another embodiment, the laser diode module 110 includes a plurality of individually packaged diode lasers, mounted on peltier coolers and each coupled to an optical fiber.

Individual diode lasers are typically packaged in TO-5 or TO-9 cans or on a C-mount. The latter option is preferred for coupling light from the diode laser into a fiber. The light coupling may be further assisted with the use of cylindrical lenses. Using such techniques, coupling efficiency between a given laser diode and its associated optical fiber may surpass 80%. In a preferred embodiment, the laser diode module 110 comprises NDV7112-E, individual laser diodes sold by Nichia, that are useful for this application. These devices have maximum optical power of 700 mW and emission wavelength between 400 and 405 nm. The operating current per laser diode is between 450 and 650 mA with a slope efficiency between 1.0 and 2.0 W/A. The operating voltage of the diodes is between 3.8 and 4.6 V, and the operating temperature is between 20 and 30° C. In one embodiment, seven individually packaged and coupled diode lasers provide up to 3.36 W of power into a fiber cable 120 having a diameter of 375 μm. In another embodiment, laser diode module 110 comprises 19 individually packaged and coupled diode lasers optically coupled to the fiber cable 120. The diode lasers are mounted such that they may receive voltage pulses simultaneously and with high efficiency at frequencies up to 1 MHz in this fashion all of the diode lasers may be made to pulse simultaneously producing optical pulses output at the optical coupler as short as 1 μs. Alternatively, the diode module 110 may comprise one diode laser optically coupled to a single optical fiber.

Delivery Fiber

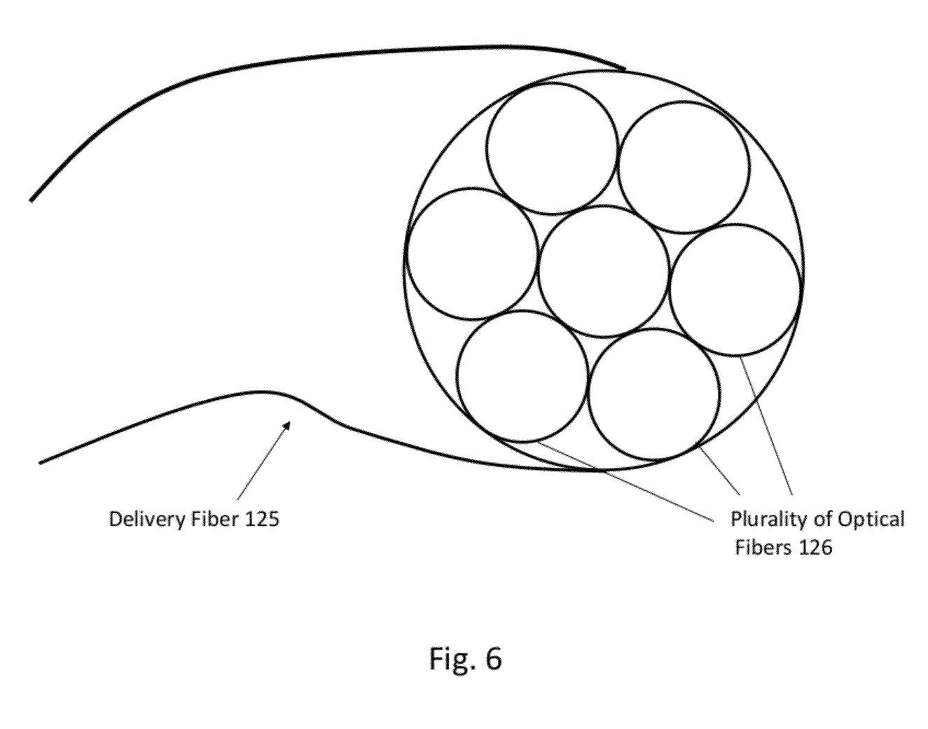

FIG. 6 illustrates an exemplary delivery fiber 125 having a plurality of optical fibers 126. The delivery fiber 125 may comprise one or more optical fibers. FIG. 6A depicts a delivery fiber 125 having a central circular optical fiber surrounding by six circular optical fibers of equal size. In a preferred embodiment, the delivery fiber comprises a central circular optical fiber surrounded by six circular optical fibers of equal size, further surrounding by 12 circular optical fibers of equal size for a total of 19 circular optical fibers of equal size. Ideally, such a fiber would include a separate laser diode optically coupled to each of the optical fibers of the delivery fiber. In another embodiment the delivery fiber has a single core 650 μm in diameter.

In another embodiment the delivery fiber has a single core 700 μm in diameter. In another embodiment the delivery fiber has a single core 1000 μm in diameter. In another embodiment, the delivery fiber 125 is a fiber cable 120 with the same number of cores as the fiber cable inside the chassis. In another embodiment, the delivery fiber is coiled to ensure mode filling. Preferably, the delivery fiber diameter is larger than the diameter of the fiber cable:

The delivery fiber may be passed into an endoscope for viewing and targeting tissue. The endoscope may include an optical filter so that the laser emission does not damage viewing systems. The optical filter may be configured to transmit light emitted by the aim diode if one is present.

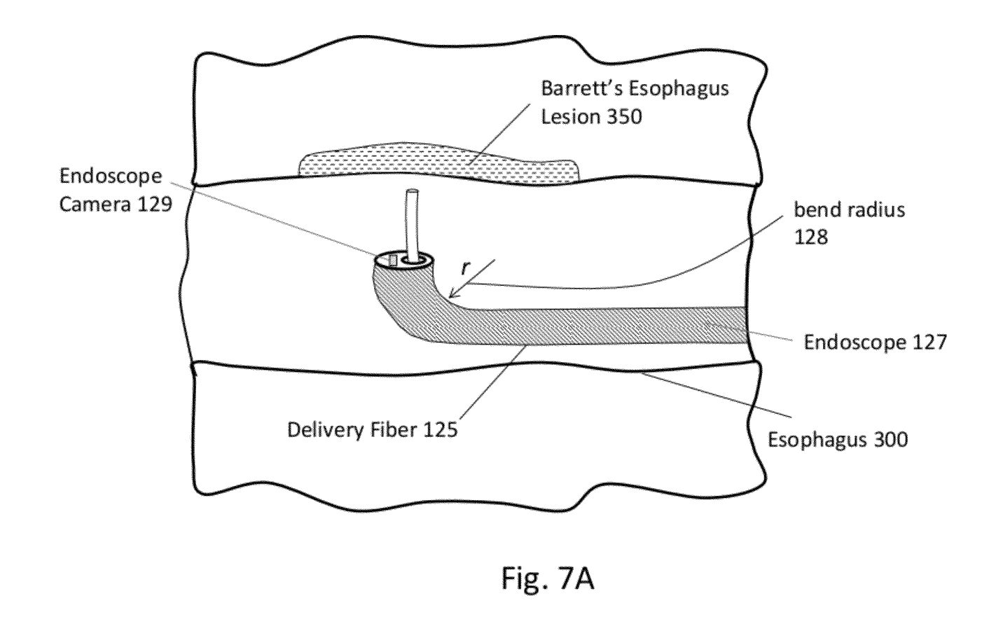

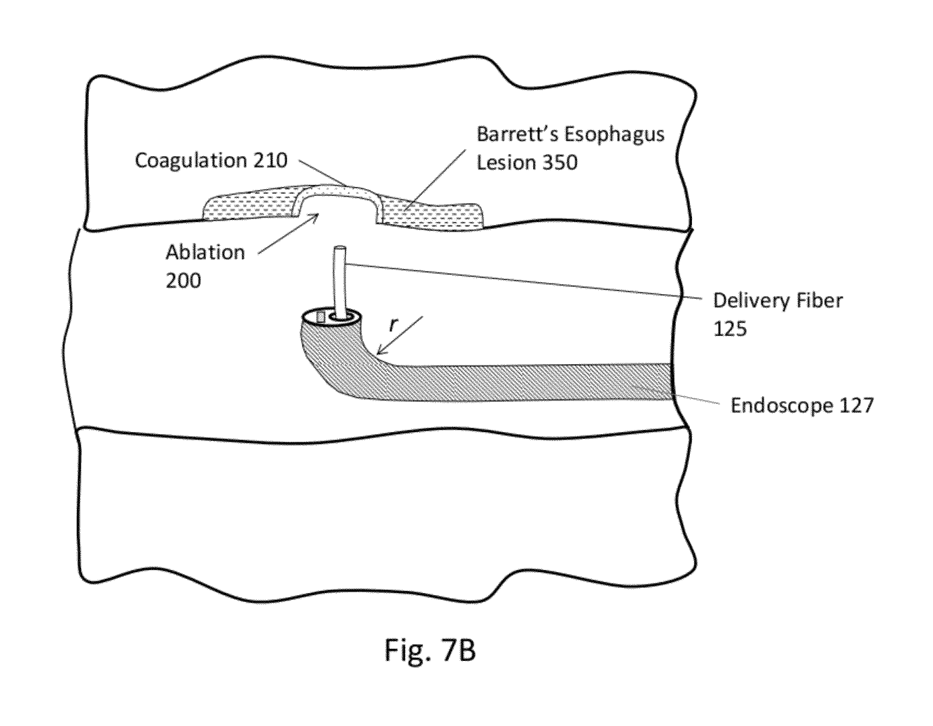

In one embodiment, the delivery fiber 125 is curved at the tip so as to direct light energy towards the inner walls of the esophagus, as shown in FIGS. 7A-7B. The curved fiber tip is oriented such that the region where the laser beam hits the wall of the esophagus is visible through the endoscope 127. In an exemplary embodiment, system 100 includes an endoscope 127 and the delivery fiber 126 can be curved by articulating the end of endoscope 127 or advancing the delivery fiber within a curved endoscope 127. Generally, the delivery fiber 126 is flexible such that the curve radius of the tip of the endoscope determines the curve of the delivery fiber tip, as shown in FIGS. 7A-7B. Typically, the endoscope also includes an endoscope camera 129 at its distal end that allows a physician to view the target treatment area during the course of the procedure. Ideally, the fiber 125 can be bent so that the distal end of the fiber can be brought to within 100 μm of the surface of the tissue to be treated. In many embodiments, the endoscope 127 or delivery fiber 125 comprise a means to rotate and translate the fiber within the esophagus such that individual areas of abnormal tissue may be treated.

The laser diode pulse parameters can be adjusted such that the fluence at the tissue is 3 kW/cm² and the pulse duration is 1 ms and the pulse repetition rate is 10 Hz. The laser diode pulse parameters can be adjusted such that the tissue damage due to coagulation is limited to a depth of 500 μm below the surface. In another embodiment, the pulse parameters and fiber positioning are adjusted such that the flux at a depth in tissue of 500 μm does not exceed 79 J/cm³ and coagulation does not occur at a depth greater than 500 μm. In another embodiment, the pulse parameters can be adjusted such that the flux does not exceed the vaporization threshold, 2019 J/cm³, anywhere in the tissue.

In some embodiments, the tissue treatment region of tissue is limited to half of the circumference of the esophagus. In another embodiment, the delivery fiber 125 contains a collimating lens to provide irradiance that varies slowly with fiber-tissue separation. The delivery fiber tip 125 may also include a diffuser with a reflector surrounding 180 degrees of the fiber such that the light emits diffusely to treat in 180 degrees. In another embodiment, the tip of the delivery fiber reflects light at a 70-90 degree angle from the axis of the fiber, and may optionally include a collimating lens to ensure the irradiance treating tissue is constant. The delivery fiber 125 may comprise a telescoping lens at the distal end to vary the irradiance at the treatment site. As shown in FIG. 7A, the tip of the delivery fiber 125 may curve or bend along a fiber bend radius 128 (r). The distal end face of the delivery fiber 125, which may be a mono-mode or multi-mode filament, can be positioned so as to direct the light energy transmitted therethrough toward the wall of the esophagus to treat an abnormal tissue, such as the Barrett's esophagus lesion 350, as shown for example in FIG. 7A. The delivery fiber 125 may be tissue treatment region, thereby creating an ablation 200 of the abnormal tissue and a coagulation 210 of the surrounding tissue and/or underlying tissue, such as the lamina propria. Coagulation 210 of this tissue at the treatment site is desirable as it may result in hemostasis which may reduce bleeding at the target treatment region.

In another embodiment, the distal end of the delivery fiber contains a photochromatic material that limits the total output power of the laser. In another embodiment, the delivery fiber has a small core return fiber that can transmit optical information such as fluorescence or scatter back to the laser module.

Coupler

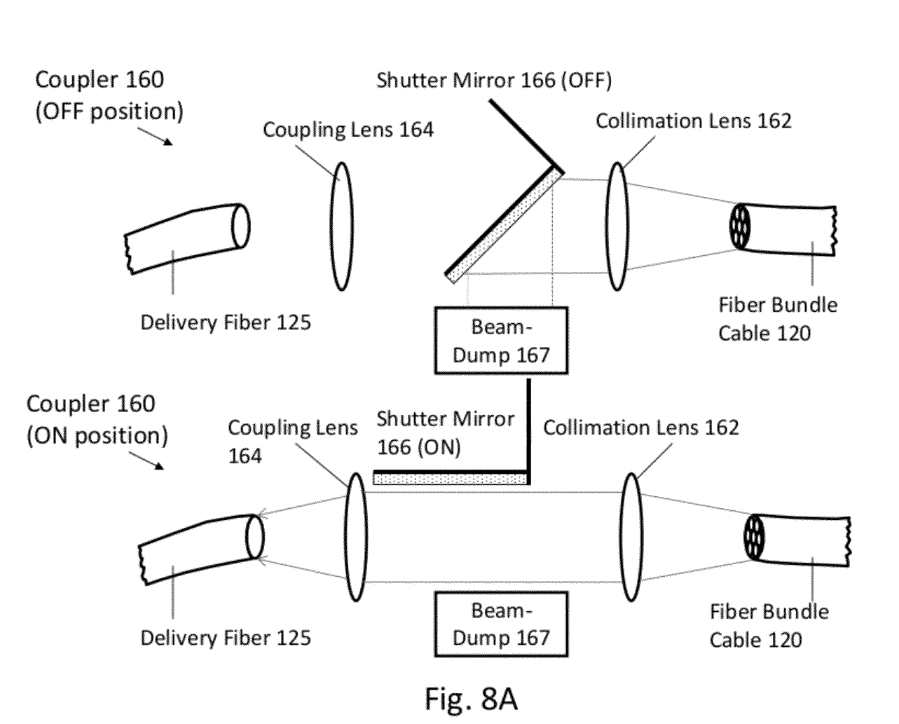

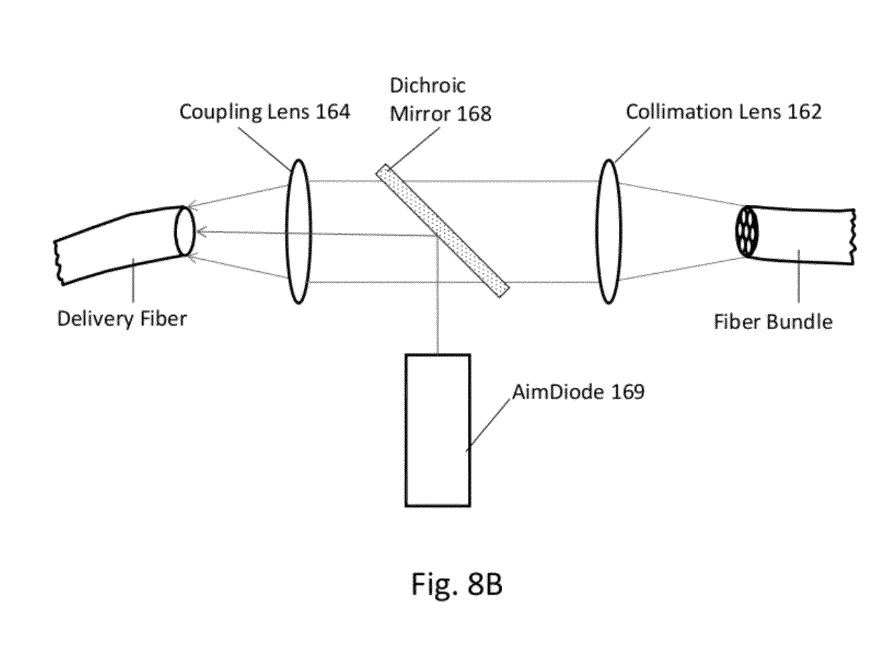

In an exemplary embodiment, the system 100 includes a coupler 160 that optically couples a distal end of fiber cable 120 to a proximal end of delivery fiber 125. Coupler 160 may further include a means to homogenize the beams emitted from the fiber cable 120. Preferably, coupler 160 includes a collimation lens 162 and a coupling lens 164, as shown for example in FIG. 8A. Collimation lens 162 has a numerical aperture greater than or equal to that of the fiber cable 120, while coupling lens 164 has a numerical aperture greater than or equal to that of the delivery fiber 125. Coupling lens 164 couples the light transmitted therethrough and projects the light energy onto the proximal end face of the delivery fiber 125. This embodiment of coupler 160 is particularly useful for transmitting light energy from a multi-mode fiber to a mono mode fiber, as shown in FIG. 8A.

In many embodiments, collimation lens 162 and coupling lens 164 are spaced sufficiently apart to allow for insertion of a shutter mirror 166 positioned and beam-dump 167. The shutter mirror 166 is movable between an ON and OFF position, as shown for example in FIG. 8A. In the ON position, the shutter mirror 166 allows light energy to be transmitted from the collimation lens 164 to the coupling lens 162 to facilitate delivery of light energy through the delivery fiber 125 during treatment. In the OFF position, the shutter mirror 166 deflects the light energy transmitted through the collimation lens 162 into beam dump 167. Moving the shutter mirror 166 between the ON and OFF position allows a user to stop and start transmission of light energy during treatment without having to activate and de-activate the laser diode module 110. The insertion of a shutter mirror 166 and beam-dump 167 increases the safety in treatment with system 100 since it allows the laser light energy to shut off completely and considerably faster than stopping the current to the laser diode module 110. Shutting the light energy off completely with shutter mirror 166 is advantageous since shutting off the light energy by changing the diode current or voltage can lead to pulses of light or very long spontaneous emission tails. Thus, shutting off the light energy “completely” with the shutter mirror 166 stops light energy from escaping , thereby preventing inadvertent treatment with light energy and improving safety during the procedure. The shutter mirror 166 and beam-dump 167 combination also allows for increased long-term reliability, since steady state laser operation is preferable over turning the laser diode module 110 on and off during treatment. Shutter mirror 166 is typically solenoid drive to be in the ON or OFF position, and preferably the mirror is driven to be either in the ON or OFF position, not in between positions. Shutter mirror 166 allows the light energy to be deflected while preventing a shutter motor that moves the mirror from overheating due to the deflected light energy. Beam-dump 167 is a thick-walled metal box with an aperture or hole to allow the deflected light energy to enter the box, thereby preventing the light energy from overheating other components of system 100.

In another embodiment, coupler 160 may includes an aim diode 169, as shown for example in FIG. 8B. An aim diode is typically a low-powered diode at a substantially different visible wavelength so that the visible light can be used to align a distal end of an optical fiber. In this embodiment, the aim diode 169 transmits a visible wavelength which is deflected by a mirror, such as dichroic mirror 168, through the coupling lens 164 and into the proximal end face of the delivery fiber 125. Typically, the light energy from the aim diode is transmitted near the center of the fiber to facilitate alignment of the distal end face of the delivery fiber 125 with the desired treatment area prior to and during the treatment process. The dichroic mirror 168 may be configured to pass light energy having a desired wavelength for use in the light energy ablation treatment while reflecting the wavelength of the light energy transmitted by aim diode 168. For example, the dichroic mirror 168 may allow light energy having a wavelength of 400-450 nm to pass through the mirror, while reflecting light energy transmitted by the aim diode 168, the aim diode 168 light energy having a wavelength between 600 and 700 nm.

As the laser diode module 110 generates significant waste heat which needs to be removed to prevent overheating, the laser diode module rests on a heat sink to absorb the excess heat. The heat sink may be cooled with a peltier cooler, refrigerated water, or blowing air.

Control Module

The control module 140 can control the operation of the system. The control module 140 contains an embedded processor and logic circuits such as an FPGA. The processor of the control module 140 comprises a tangible medium having instructions of a computer readable program embodied thereon. The control module 140 may comprise programmable read-only-memory which further comprises the programs to implement algorithms which define system operation. The control module comprises means to receive information in the form of voltage signals from the user interface 150, the footpedal 142, temperature monitors, the laser diode module 110 and a card reader for security card 144. The control module 140 further comprises means to transmit information in the form of voltage signals to the DC power supply 170, the pulse generator 130, the laser diode module 110 and the user interface 150. The control module 140 comprises a means to receive DC power. The control module 140 also comprises the means to evaluate security codes transmitted from the security card 144.

Pulse Generator

The pulse generator 130 comprises a frequency reference, for example a timer, and a means to generate voltage pulses. The pulse generator 130 receives encoded instructions from the control module. The instructions contain information describing the desired amplitude of the voltage pulse, the duration of the voltage pulse and the repetition frequency of the voltage pulse. The pulse generator 130 comprises the means to generate voltage pulses at frequencies from 1 mHz to 1 MHz. The pulse generator 130 comprises the means to generate pulses that are square, that are sinusoidal or that are saw-toothed. The pulse generator 130 comprises the means to generate pulses with peak amplitudes of 4.5 V. The pulse generator 130 further comprises a means to transmit the voltage pulses to the laser diode module. The pulse generator 130 comprises a means to transmit its status conditions to the control module.

User Interface

The user interface 150 comprises a means to receive information from the user. In one embodiment the user interface is keyboard and display. In another embodiment, the user interface 150 is a screen, such as a touchscreen or a screen having a cursor controlled by direction keys or a mouse. The user interface 150 further comprises a means for transmitting signals to the control module. The user interface 150 further comprises a means of receiving signals from the control module. The user interface 150 comprises a means for the user to select pulse duration, pulse energy, pulse frequency. The user interface 150 comprises a means to display system status information such as error conditions, usage rates, or laser emission.

DC Power Supply

The DC power supply 170 comprises a means to receive AC power from outside the system. The DC power supply 170 comprises a means to convert AC power to DC power. In one embodiment the DC power supply 170 converts 110 V AC power to 24 V DC power. In another embodiment, the DC power supply 170 provides multiple DC voltages, said voltages transmitted to and used to power the other system modules.

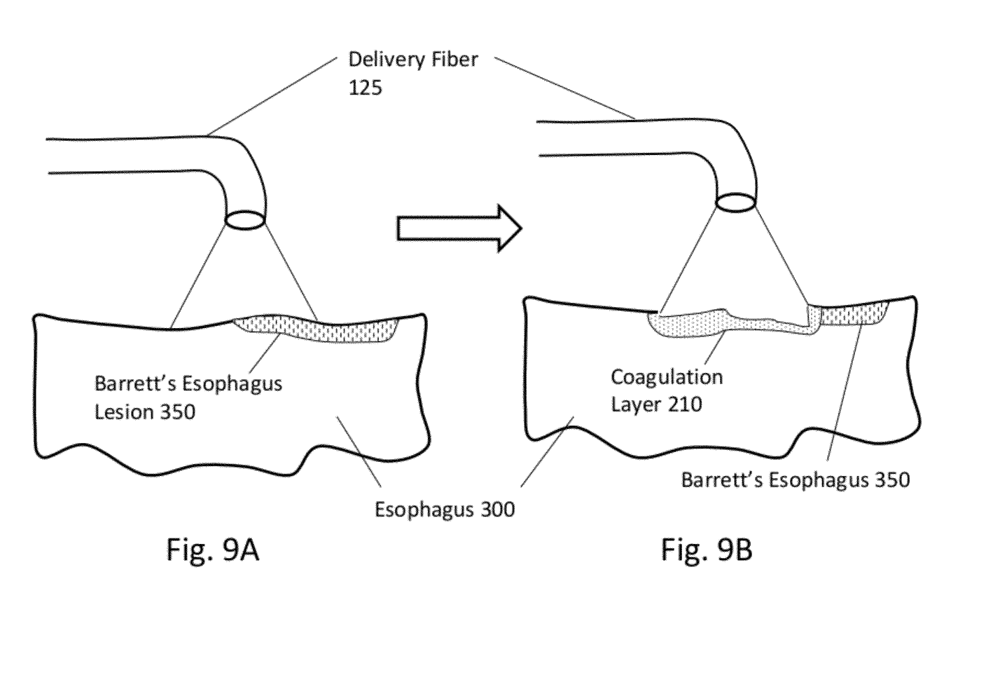

The system 100 as described herein can be configured in many ways to treat tissue. For example, as shown in FIGS. 9A-9B, the laser beam transmitted through delivery fiber 125 can be configured to ablate a BE lesion 350 and coagulate normal tissue creating coagulation layer 210. Alternatively or in combination, the laser beam can be adjusted so as to ablate the BE lesion tissue such coagulation of the normal tissue exposed to the beam is substantially minimized. Alternatively or in combination, the laser beam can be adjusted such that the Barrett's esophagus tissue is coagulated with the laser beam and the normal tissue is substantially not coagulated with similar exposure to the laser beam. Thus, by using empirically determined treatment parameter, a user can selectively treat the BE lesion tissue as desired with the above described system.

The above describes specific embodiments in accordance with embodiments described herein. A person of ordinary skill in the art will recognize various modifications based on the teachings described herein. Therefore, the scope of the present invention is limited solely by the claims and the full scope of their equivalents.

A two-phase study has been completed to provide a proof-of-concept evaluation of the above described system and methods for treating BE. In the first phase of the study, a bench-top evaluation of treatment parameters was performed on ex vivo porcine tissue samples. The second phase was an acute in vivo study performed on a single pig.

In these studies, system 100 comprised a 6 W Nichia NUV101E diode laser module, a DC power supply, signal generation, cooling and performance. Laser performance parameters were measured. The power out of the fiber as a function of drive voltage was measured showing a peak power of over 6 W. The response time of the system 100 to pulsing of the drive voltage was measured showing a rise time of approximately 200 μs thus constraining measurements to pulse durations of 2 ms or longer.

For both phases of the study, an 800-μm diameter fiber enabled targeted delivery of the laser light to the tissue samples. The slowly expanding beam provided by the 0.22 numerical aperture of the fiber provided a spot size that could be controlled by controlling the fiber-tissue separation. At 500 μm separation, the spot size was 1020 μm resulting in a maximum energy flux of 734 W/cm².

Tissue tests were performed on the ex vivo porcine esophagus and small intestine tissue. The laser was driven by a voltage pulse train. The peak voltage of the pulses determined the peak power of the laser as calibrated by earlier power measurements, and the pulse train duty cycle, frequency and duration were monitored electronically. All parameters were evaluated for their impact on tissue effect. Different portions of the ex vivo porcine esophagus were treated with system 100 at differing flux. After treatment, the tissue was sliced transverse to the damage tracks and evaluated under a microscope.

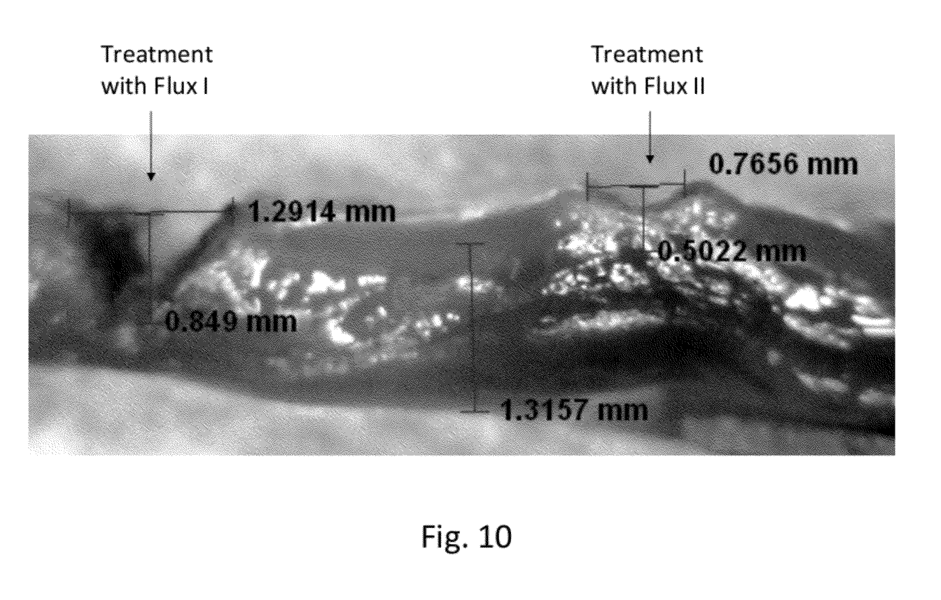

The tissue was thawed and allowed to rise to room temperature. Both the esophagus and a section of the small intestine were filleted open and 25-cm sections were sliced off. The slices were laid on a thick glass slide for heat sinking. Mechanical fixturing and a stepper motor were used to ensure that a minimal fiber-tissue separation was maintained and consistent dosages applied. An estimate of the appropriate flux for in vivo treatment was made by measuring the ablation depth at the portions having been treated at differing flux. As shown in FIG. 10, a representative measurement from the treated portions of the small intestine target. The ablated portion having been treated at Flux I was measured as having a width of 1.2914 mm and a depth of 0.849 mm. The ablated portion having been treated at Flux II was measured as having a width of 0.7656 mm and a depth of 0.5022 mm. As the squamous epithelium is roughly about 0.5 mm in thickness, a treatment depth of 500 μm is ideal for the ablation of Barrett's esophagus. Thus, Flux II, the flux used to create the channel on the right-hand-side of FIG. 10 was selected for the in vivo animal study.

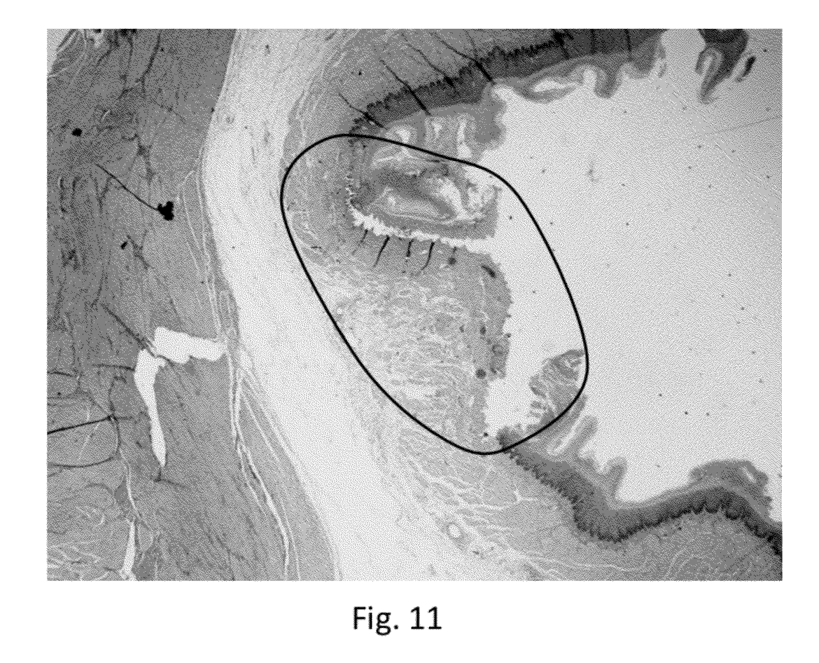

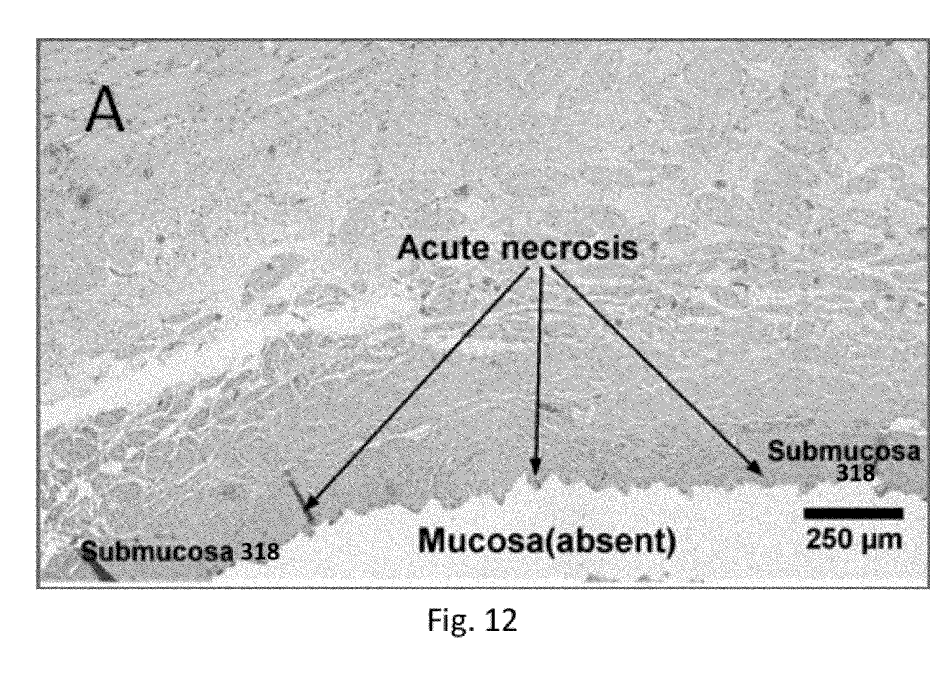

For the in vivo animal study, a single Yorkshire pig was selected for an acute study with an IACUC-approved protocol. Several test treatments wete taken from inside the stomach to verify system performance and reduce the fluence to accommodate the stronger absorption due to the presence of oxygenated hemoglobin in an in vivo model. Three separate sites along the distal esophagus were selected for treatment using differing laser pulse parameters. The pig was euthanized immediately following the treatment, and the treatment sites were extracted for histology. A representative histology sample from the esophagus is shown in FIGS. 11-12.

FIG. 11 shows an esophageal wall of the pig after laser treatment, the outlined area having been exposed to the light energy treatment of system 100. The relative thickness of the various layers in the esophageal wall are visible at top, the squamous epithelium having been ablated within the outlined area.

FIG. 12 shows a magnified image of the ablated area in a tissue treatment region in the porcine esophagus. The squamous epithelium layer is noticeably absent from the lining of the treated porcine esophageal wall. The submucosa beneath (shown by the arrows) sustained marked tissue necrosis which extends into the underlying muscularis layer.

The evaluation of the histology results from the animal study showed that system 100 was able to ablate the squamous epithelium and deliver thermal damage into the lower layers of the esophagus in a manner sufficient to treat Barrett's esophagus lesions in an esophagus. The flux at the tissue required to achieve this level of damage in our tests was 244 W/cm². The flux exiting the fiber was 384 W/cm². The total energy fluence delivered to tissue was 195 J/cm². The results further showed that the thermal damage extended laterally for several hundred micrometers on either side of the treatment area. Both of these findings are consistent with the benchtop observations. The laser fluence used in the in vivo tests was less than predicted by the benchtop studies because of the absence of oxygen in ex vivo tissue samples. The histology combined with the measurement of laser performance for the parameters in use during treatment allowed for the determination of an optimum fluence for the treatment of Barrett's esophagus. Optimum treatment of Barrett's esophagus occurs when the squamous epithelium is ablated (about 0.5 to 1 mm in depth), coagulation for hemostasis extends approximately another 0.5 to 1 mm into the muscularis mucosae (extending another 2-3 mm), and thermal damage is limited to the muscularis mucosa such that the submucosa is relatively undamaged (3 mm or deeper).

What is claimed is:

1. A system to treat tissue comprising:
at least one laser diode to emit light energy; and
at least one optical fiber to deliver light energy to the tissue.

2. The system of claim 1 wherein the tissue comprises Barrett's esophagus tissue and wherein the light energy comprises a wavelength within a range from about 360 nm to about 450 nm.

3. The system of claim 1 wherein the light energy comprises a wavelength range from about 400 nm to about 430 nm.

4. The system of claim 1 wherein the light energy comprises a wavelength range from about 400 nm to about 410 nm.

5. The system of claim 1 wherein the output power of the system delivered to the tissue is at least about 10 W.

6. The system of claim 1 wherein an optical light flux energy exiting the at least one fiber corresponds to an optical light flux energy on a surface of the tissue within a range from about 100 W/cm² to about 400 W/cm².

7. The system of claim 6 wherein the optical light flux energy exiting the at least one fiber and the optical light flux energy are each within the range from about 100 W/cm² to about 400 W/cm² when a distal end of the at least one fiber is placed within about 1 mm of the surface of the tissue.

8. The system of claim 6 wherein the optical light flux energy is delivered for a cumulative amount of time corresponding to fluence per unit area within a range from about 100 to about 300 J/cm² such that a first layer having Barrett's esophagus tissue comprising hemoglobin is ablated and an underlying layer comprising a lamina propria is coagulated.

9. The system of claim 1 further comprising:
a shutter located along an optical path extending between the at least one laser diode and the tissue; and
circuitry coupled to the shutter to open and close the shutter such that the shutter allows passage of the light energy along the optical path to treat the tissue when open and the shutter prevents passage of the light energy to the tissue when closed.

10. The system of claim 2, wherein the light energy comprises one or more of a pulsed beam and a substantially continuous beam.

11. The system of claim 10 further comprising:
a pulse generator, wherein the pulse generator is configured to provide the pulsed beam such that each pulse has a duration within a range from about 5 ms to about 100 ms corresponding to a duty cycle within a range from about 70% to about 95%.

12. The system of claim 10 wherein the at least one laser diode comprises a plurality of laser diodes and wherein the at least one optical fiber comprises a plurality of first optical fibers and a second multimode optical fiber, each of the first plurality of optical fibers having a first end and a second end,
wherein said first end of each of the first plurality of optical fibers is optically coupled to one of the plurality of laser diodes, and
wherein said second end of each of the first plurality of optical fibers is optically coupled to the second multimode optical fiber so as to smooth a laser energy output beam profile of the light energy delivered from the multimode optical fiber to the tissue.

13. The system of claim 12 further comprising at least one lens to optically couple the first plurality of optical fibers to the second multimode optical fiber, the at least one lens placed between said second end of each of the first plurality of optical fibers and a first end of the second multi-mode optical fiber such that the at least one lens forms an image of the second end of each of the first plurality of optical fibers on the first end of the second multi-mode optical fiber.

14. The system of claim 13 wherein the at least one lens comprises a first lens having a first focal length and a second lens having a second focal length, the first lens separated from the second end of the first plurality of optical fibers with a first distance corresponding to the first focal length, the second lens separated from the first end of the second multi-mode optical fiber with a second distance corresponding to the second focal length such that the laser beam extends substantially collimated between the first lens and the second lens, the system further comprising a shutter located between the first lens and the second lens.

15. A method of treating tissue, comprising:
generating light energy with at least one diode laser; and
treating the tissue with the light energy.

16. The method of claim 15 wherein the tissue comprises esophageal tissue having a Barrett's esophagus lesion covering a lamina propria, the method further comprising introducing at least one optical fiber into the esophagus such that a distal end of the at least one optical fiber is located within about 1 mm of the lesion, wherein the light energy is delivered to the tissue so as to remove at least a portion of the Barrett's esophagus lesion with ablation so as to expose and coagulate at least a portion of the lamina propria.

17. The method of claim 16 wherein the ablated portion of the lesion removed with the light energy comprises a first layer having a thickness within a range from about 250 um to about 0.75 um and wherein the portion of the lamina propria comprises a layer having a thickness within a range from about 250 um to about 750 um.

18. The method of claim 17 wherein the distal end of the optical fiber is moved so as to scan the light energy over a treatment area of the esophagus, the treatment area having a maximum dimension across at least twice that of a maximum dimension across the at least one optical fiber.

19. The method of claim 15 wherein the tissue comprises hemoglobin and the light energy comprises a wavelength within a range from about 360 nm to about 450 nm.

20. The method of claim 15 wherein the tissue comprises hemoglobin and the light energy comprises a wavelength within a range from about 400 nm to about 430 nm.

21. The method of claim 15 wherein the tissue comprises hemoglobin and the light energy comprises a wavelength within a range from about 400 nm to about 410 nm.

22. The method of claim 15 wherein the flux at a surface of the tissue is between 100 and 500 W/cm².

23. The method of claim 15 wherein the energy fluence at tissue is between 200 and 300 J/cm².

24. The method of claim 15 wherein the tissue comprises esophageal tissue having a first portion and a second portion, the first portion comprising a Barrett's esophagus tissue, the second portion comprising a lamina propria of the esophagus, and wherein the Barrett's esophagus tissue is removed with ablation and the second portion comprising the lamina propria portion is coagulated.

25. The method of claim 15 wherein the optical light flux energy is delivered for a cumulative amount of time corresponding to fluence per unit area within a range from about 100 to about 300 J/cm² such that a first layer having Barrett's esophagus tissue comprising hemoglobin is ablated and an underlying layer comprising a lamina propria is coagulated.
